(19) United States
(12) Patent Application Publication
(10) Pub. No.: US 2025/0020551 A1
(43) Pub. Date: Jan. 16, 2025

(54) GAS COLLECTION DEVICE

(71) Applicant: LG ENERGY SOLUTION, LTD., Seoul (KR)

(72) Inventors: Kyung Min KIM, Daejeon (KR); Nak Hee CHOI, Daejeon (KR)

(21) Appl. No.: 18/714,526

(22) PCT Filed: Jul. 21, 2023
(86) PCT No.: PCT/KR2023/010584

§ 371 Date: May 29, 2024

(30) Foreign Application Priority Data
Jul. 28, 2022 (KR) 10-2022-0094123

(51) Int. Cl. G01N 1/22; H01M 10/42

(52) U.S. Cl. CPC G01N 1/2226; H01M 10/4285

(57) ABSTRACT

Provided are gas collection devices. Such gas collection devices may be capable of providing improved gas concentrations for an analysis device by controlling the space where gas is diffused when collecting gas generated from a battery.

### CROSS-REFERENCE TO RELATED APPLICATIONS

This application is a National Phase entry pursuant to 35 U.S.C. § 371 of International Application No. PCT/KR2023/010584 filed on Jul. 21, 2023, which claims priority to and the benefit of Korean Patent Application No. KR 10-2022-0094123, filed on Jul. 28, 2022. The contents of the above-identified applications are herein incorporated by reference in their entireties.

### TECHNICAL FIELD

The present disclosure relates to a gas collecting apparatus, and particularly to a gas collecting apparatus capable of providing improved gas concentrations to an analysis device by controlling a space in which gas generated in a battery is diffused in a process of collecting the gas.

### BACKGROUND

In general, a secondary battery is a battery which can be used repeatedly through a discharging process in which chemical energy is converted into electrical energy and a charging process in the opposite direction to the discharging process and which includes nickel-cadmium (Ni—Cd) batteries, nickel-metal hydride (Ni-MH) batteries, lithium-metal batteries, lithium-ion (Li-ion) batteries, and lithium-ion polymer batteries. Among these secondary batteries, lithium secondary batteries having high energy density and voltage, long cycle life, and a low self-discharge rate have been commercialized and widely used.

Depending on the reaction within the lithium secondary battery, various types of gases such as hydrogen, oxygen, nitrogen, carbon monoxide, carbon dioxide, hydrocarbons of CₙH₂ₙ₋₂ (n=2˜5), CₙH₂ₙ (n=2˜5), CₙH₂ₙ₊₂ (n=1˜5), and other organic gas species, may be generated.

In addition, the lithium secondary battery degrades while generating a large amount of gas due to electrolyte decomposition as repeated charging and discharging progresses, and this aspect appears differently depending on the design and use form of the battery. Therefore, it is essential to infer a deterioration mechanism of a battery by analyzing gas generated inside the battery during a battery development process.

Therefore, it is very important to collect and accurately analyze the gas generated in the secondary battery. Various gases are generated during the operation of lithium ion batteries, and information on the composition and content of generated gases is useful for developing battery materials, optimizing battery manufacturing processes, and identifying causes of battery defects. To this end, it is important to develop a technology to collect the gas generated inside the secondary battery.

As one method for collecting gas generated from the secondary battery, after placing the secondary battery in an airtight diffusion space and depressurizing the secondary battery, a hole was made in the secondary battery to diffuse the generated gas into the diffusion space accommodating the secondary battery, and samples were taken.

Specifically, a gas collecting container or a gas analyzer in which a vacuum was formed was connected to the diffusion space, and the gas diffused in the diffusion space was delivered to the gas collecting container or the gas analyzer. Accordingly, the concentration of the finally collected gas varied depending on the size of the diffusion space or the specification (size) of the battery.

Since the concentration of the collected gas not only affects detection sensitivity of the analysis device but is also an important variable for calculating quantitative data, a method for precisely controlling it is required.

The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY

The present disclosure relates to a gas collecting apparatus, and is to provide a gas collecting apparatus capable of providing improved gas concentrations to an analysis device by controlling a space in which gas generated in a battery is diffused in the process of collecting the gas.

Technical objects to be achieved by the present disclosure are not limited to the technical objects mentioned above, and other technical objects not mentioned will be clearly understood by those skilled in the art from the description below.

A gas collecting apparatus of the present disclosure may include
a gas diffusion chamber including a gas diffusion space;
a variable dilution tank connected to the gas diffusion chamber and including a dilution space;
a volume control unit configured to control a volume of the dilution space of the variable dilution tank;
a punching unit configured to form a perforated hole in a case of a battery insertable into the gas diffusion space of the gas diffusion chamber; and
a gas delivery flow path connected to the gas diffusion chamber to receive gas diffused into the gas diffusion space and deliver the gas to a gas collecting container or a gas analyzer.

In the gas collecting apparatus of the present disclosure, the volume control unit may include a moving unit located inside the variable dilution tank and configured to move in a vertical direction, a shaft coupled to an upper surface of the moving unit, wherein a lower end of the shaft is inserted inside the variable dilution tank through a penetration hole on an upper surface of the variable dilution tank, the shaft configured to move in the vertical direction, and a first driving unit configured to rotate the shaft with the vertical direction as a rotation axis.

In the gas collecting apparatus of the present disclosure, the gas diffusion chamber may include a lower jig with a battery accommodating groove on an upper surface of the lower jig as the gas diffusion space, and an upper jig coupled to the upper surface of the lower jig while covering the battery accommodating groove, wherein a first hole is on an upper surface of the upper jig, a lower end of the variable dilution tank is coupled to the upper surface of the upper jig while covering the first hole, and gas in the gas diffusion space and gas in the dilution space are mutually ventilated through the first hole.

In the gas collecting apparatus of the present disclosure, the volume control unit may further include a support unit configured to fix a relative distance between the first driving unit and the upper jig.

In the gas collecting apparatus of the present disclosure, the volume control unit may further include a measuring unit configured to measure a moving distance of the moving unit in the vertical direction.

In the gas collecting apparatus of the present disclosure, the measuring unit may include a moving member configured to move in the vertical direction along with the shaft, a fixing member coupled to the upper surface of the upper jig, and a display member configured to indicate a displacement between the moving member and the fixing member.

In the gas collecting apparatus of the present disclosure, an inner space of the variable dilution tank may have a column shape extending in the vertical direction, a side surface of the moving unit may be in contact with an inner circumferential surface of the variable dilution tank, and the dilution space may be a space located at a lower side of the moving unit in the inner space of the variable dilution tank.

In the gas collecting apparatus of the present disclosure, a sealing cover surrounding the shaft may be located at an upper side of the moving unit in the inner space of the variable dilution tank, and the sealing cover may be contracted or expanded in the vertical direction.

In the gas collecting apparatus of the present disclosure, an upper end of the sealing cover may be coupled to a ceiling surface of the variable dilution tank in the inner space of the variable dilution tank, and a lower end of the sealing cover may be coupled to the upper surface of the moving unit.

In the gas collecting apparatus of the present disclosure, a second hole for inserting a punching needle into the gas diffusion space may be on the upper surface of the upper jig, and the punching unit may include a punching needle housing coupled to the upper surface of the upper jig with a lower end of the punching needle housing covering the second hole and configured to accommodate the punching needle inside, a rod with a lower end of the rod coupled to the punching needle inside the punching needle housing, a second driving unit coupled to an upper end of the rod and configured to move the rod in the vertical direction, and a fixing unit configured to fix a position between the punching needle housing and the second driving unit.

A gas collecting apparatus of the present disclosure may be capable of collecting a gas with a constant concentration in a gas collecting container by providing a controllable variable space to a gas diffusion space.

The gas collecting apparatus of the present disclosure may provide an improved gas concentration to an analysis device by controlling a space in which gas generated from a battery is diffused in the process of collecting the gas.

### BRIEF DESCRIPTION OF THE DRAWINGS

The accompanying drawings illustrate various embodiments of the present disclosure and together with the foregoing disclosure, serve to provide further understanding of the technical features of the present disclosure, and thus, the present disclosure is not construed as being limited to the drawings.

FIG. 1 is a perspective view illustrating a gas collecting apparatus according to the present disclosure.

FIG. 2 is a perspective view illustrating a state in which a gas diffusion chamber is opened.

FIG. 3 is a perspective view illustrating a variable dilution tank and a volume control unit.

FIG. 4A is a cross-sectional view illustrating a cross section A-A′ of FIG. 3.

FIG. 4B is a cross-sectional view illustrating a state in which a dilution space is expanded.

FIG. 5 is a perspective view illustrating a punching unit.

### DETAILED DESCRIPTION

Hereinafter, embodiments according to the present disclosure will be described in detail with reference to the accompanying drawings. In this process, the size or shape of the components shown in the drawings may be exaggerated for clarity and convenience of explanation. In addition, terms specifically defined in consideration of the configuration and operation of the present disclosure may vary according to the intentions or customs of users and operators. Definitions of these terms should be made based on the content throughout this specification.

In the description of the present disclosure, it should be noted that the orientation or positional relationship indicated by the terms “center”, “upper”, “lower”, “left”, “right”, “vertical”, “horizontal”, “inner side”, “outer side”, “one surface”, “other surface” is based on the orientation or positional relationship shown in the drawing or the orientation or positional relationship normally arranged when using the product of the present disclosure, and it is intended only for explanation and brief description of the present disclosure, and is not to be construed as limiting the present disclosure as it does not suggest or imply that the device or element shown must necessarily be configured or operated in a specific orientation with a specific orientation.

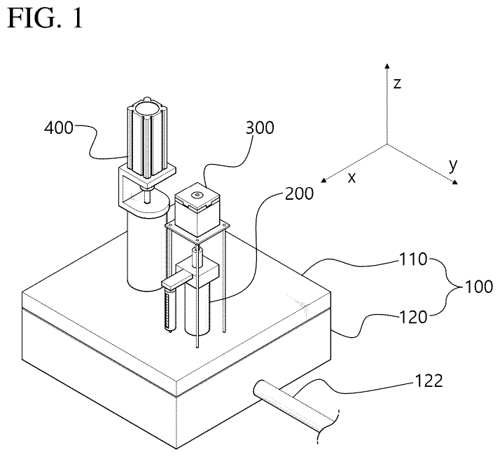

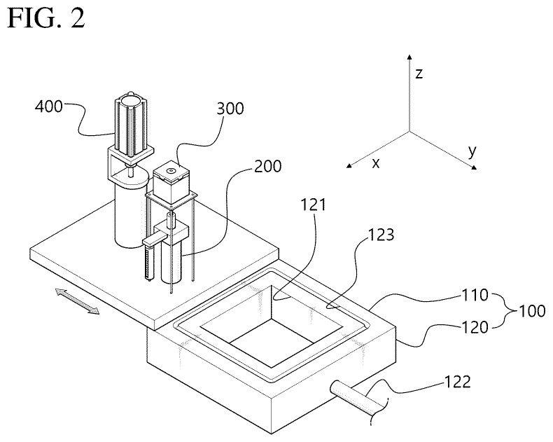

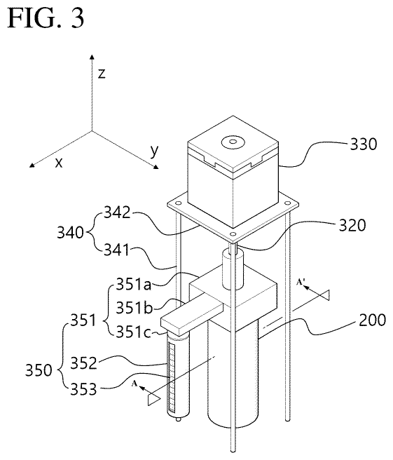

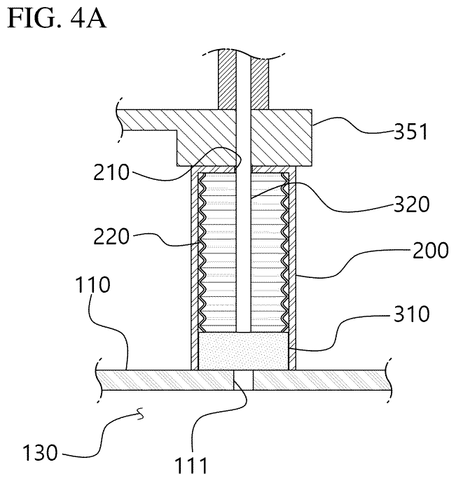

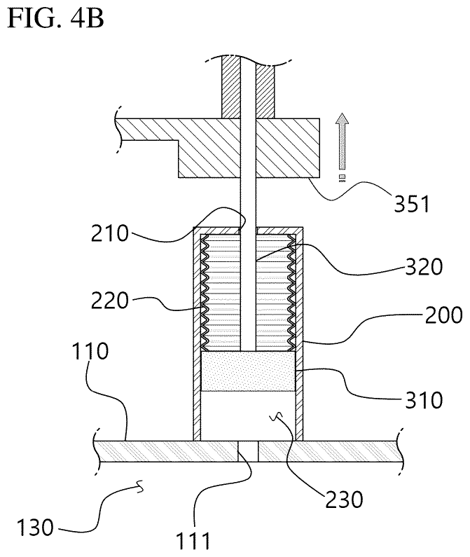

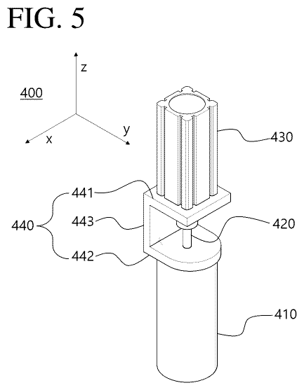

FIG. 1 is a perspective view illustrating a gas collecting apparatus according to the present disclosure. FIG. 2 is a perspective view illustrating a state in which a gas diffusion chamber 100 is opened. FIG. 3 is a perspective view illustrating a variable dilution tank 200 and a volume control unit 300. FIG. 4A is a cross-sectional view illustrating a cross section A-A′ of FIG. 3. FIG. 4B is a cross-sectional view illustrating a state in which a dilution space 230 is expanded. FIG. 5 is a perspective view illustrating a punching unit 400.

Hereinafter, the gas collecting apparatus of the present disclosure will be described in detail with reference to FIGS. 1 to 5.

As shown in FIG. 1, the gas collecting apparatus of the present disclosure may include
the gas diffusion chamber 100 provided with a gas diffusion space 130 inside;
the variable dilution tank 200 connected to the gas diffusion chamber 100 and provided with the dilution space inside 230;
the volume control unit 300 configured to control a volume of the dilution space 230 of the variable dilution tank 200;
the punching unit 400 configured to form a perforated hole in a case of a battery accommodated in the gas diffusion space 130 of the gas diffusion chamber 100; and
a gas delivery flow path 122 connected to the gas diffusion chamber 100 to receive gas diffused in the gas diffusion space 130 and deliver the gas to a gas collecting container or a gas analyzer.

Gas capture using the gas capture apparatus of the present disclosure may be accomplished as follows. First, the battery is accommodated in the gas diffusion space 130 provided inside the gas diffusion chamber 100. A perforated hole is formed in the battery case through the use of punching unit 400 while the battery is accommodated in the gas diffusion space 130. When the gas generated inside the battery is discharged out of the battery through the perforated hole, the battery generated gas is diffused into the gas diffusion space 130 of the gas diffusion chamber 100. The pressure of the gas diffusion space 130 is measured, and when the pressure is greater than the set pressure, the volume control unit 300 is operated to expand the dilution space 230 of the variable dilution tank 200. When the pressure of the gas diffusion space 130 is lower than the set pressure, a gas collecting container or a gas analyzer may be connected to the gas delivery flow path 122 to collect the battery generated gas.

As shown in FIGS. 1 and 2, the gas diffusion chamber 100 may include a lower jig 120 with a battery accommodating groove 121 formed on an upper surface as the gas diffusion space 130, and an upper jig 110 coupled to an upper surface of the lower jig 120 while covering the battery accommodating groove 121.

The upper jig 110 may be provided in a plate shape, and may be coupled to the lower jig 120 such that a lower surface of the upper jig 110 contacts the upper end of the lower jig 120. The variable dilution tank 200, the volume control unit 300, and the punching unit 400 may be fixed to the upper surface of the upper jig 110. A first hole 111 and a second hole (not shown) may be formed in the upper jig 110. The variable dilution tank 200 may be fixed to the upper surface of the upper jig 110 while covering the first hole 111 and the punching unit 400 may be fixed to the upper surface of the upper jig 110 while covering the second hole. The first hole 111 may be a passage through which the gas in the gas diffusion space 130 is delivered to the dilution space 230, and the second hole may be a passage through which the punching unit 400 inserts the punching needle into the gas diffusion space 130.

In other words, a first hole 111 is formed on an upper surface of the upper jig 110, a lower end of the variable dilution tank 200 is coupled to the upper surface of the upper jig 110 while covering the first hole 111, and gas in the gas diffusion space 130 and gas in the dilution space 230 are mutually ventilated through the first hole 111. At this time, the volume of the dilution space 230 is varied by the volume control unit 300, and concentration control of the gas diffused in the gas diffusion space 130 may be achieved by controlling the volume of the dilution space 230.

On the upper surface of the lower jig 120, a battery accommodating groove 121 provided to the gas diffusion space 130 may be provided. On the upper surface of the lower jig 120, a sealing member insertion groove 123 may be provided so that when the upper jig 110 and the lower jig 120 are coupled, the sealing member is inserted into the sealing member insertion groove 123 to enhance the airtightness of the gas diffusion space 130. The sealing member and the sealing member insertion groove 123 are provided in a closed loop shape, and the battery insertion groove may be located inside the sealing member insertion groove 123. For example, a sealing member may be an O-ring.

As shown in FIG. 2, the upper jig 110 may be coupled with or separated from the lower jig 120 while sliding in the y-axis direction. The upper jig 110 may linearly move in the y-axis direction by a pneumatic cylinder (not shown).

The gas delivery flow path 122 may be a chemical resistant pipe. The gas delivery flow path 122 may be provided with an opening/closing valve for discharging or blocking the gas in the gas diffusion space 130. A manifold may be connected to the gas delivery flow path 122, and a pressure sensor may be connected to the manifold to measure the pressure of the gas diffusion space 130.

As shown in FIGS. 3 and 4A, the volume control unit 300 may include a moving unit 310 which is located inside the variable dilution tank 200 and moves in a vertical direction, a shaft 320 coupled to an upper surface of the moving unit 310 in a state in which a lower end is inserted inside the variable dilution tank 200 through a penetration hole 210 provided on an upper surface of the variable dilution tank 200 and moving in the vertical direction, and a first driving unit 330 configured to rotate the shaft 320 with the vertical direction as a rotation axis.

The moving unit 310 may adjust the volume of the dilution space 230 by moving in the inner space of the variable dilution tank 200 while separating the inner space of the variable dilution tank 200 into two spaces.

Specifically, an inner space of the variable dilution tank 200 may be provided in a column shape extending in the vertical direction, a side surface of the moving unit 310 may be in close contact with an inner circumferential surface of the variable dilution tank 200 in the inner space of the variable dilution tank 200, and the dilution space 230 may be a space located at a lower side of the moving unit 310 in the inner space of the variable dilution tank 200. For example, the dilution space 230 and the moving unit 310 of the variable dilution tank 200 may be provided in a cylindrical shape extending in the vertical direction, and the outer circumferential surface of the moving unit 310 may be in close contact with the inner circumferential surface of the variable dilution tank 200.

As shown in FIG. 4B, when the moving unit 310 is raised by the volume control unit 300, the dilution space 230 of the variable dilution tank 200 may be expanded, and the gas concentration in the gas diffusion space 130 may be controlled. The gas collecting apparatus of the present disclosure may precisely control the movement of the moving unit 310 by converting rotational force into a linear driving force to move the moving unit 310.

As shown in FIGS. 4A and 4B, a sealing cover 220 surrounding the shaft 320 may be provided at an upper side of the moving unit 310 in the inner space of the variable dilution tank 200, and the sealing cover 220 may be contracted or expanded in the vertical direction. An upper end of the sealing cover 220 may be coupled to a ceiling surface of the variable dilution tank 200 in the inner space of the variable dilution tank 200, and a lower end of the sealing cover 220 may be coupled to the upper surface of the moving unit 310. The sealing cover 220 not only prevents gas leaking to the side of the moving unit 310 from being discharged to the outside through the penetration hole 210, but also may prevent gas from being diffused into spaces other than the dilution space 230 in the inner space of the variable dilution tank 200. For example, the outer circumferential surface of the sealing cover 220 is formed to be as close as possible to the inner circumferential surface of the variable dilution tank 200, so that even if the lower gas of the moving unit 310 leaks upward, the volume in which gas is substantially diffused inside the variable dilution tank 200 may be approximated as a volume of the dilution space 230. The upper and lower ends of the sealing cover 220 may be welded to a ceiling surface of the variable dilution tank 200 and the upper surface of the moving unit 310, respectively.

The shaft 320 may be provided in a cylindrical shape extending in the vertical direction and a screw thread may be formed on an outer circumferential surface of the shaft 320. The shaft 320 may be screw-coupled to the variable dilution tank 200 or a structure having a fixed location relative to the variable dilution tank 200. Accordingly, the shaft 320 may move in the vertical direction with respect to the variable dilution tank 200 by rotating. For example, a screw thread may be provided on the inner circumferential surface of the penetration hole 210, and by rotating the shaft 320 in a state where the thread of the penetration hole 210 and the thread of the shaft 320 are coupled, the shaft 320 may move in the vertical direction with respect to the variable dilution tank 200. The lower end of the shaft 320 is coupled to the moving unit 310 so as to be rotatable so that the moving unit 310 can be prevented from rotating together when the shaft 320 rotates.

The first driving unit 330 may rotate the shaft 320 with the vertical direction as a rotation axis. The first driving unit 330 may be an electric motor.

The volume control unit 300 may further include a support unit 340 for fixing a relative distance between the first driving part 330 and the upper jig 110.

As shown in FIG. 3, the support unit 340 may include a support plate 342 located on the upper part of the variable dilution tank 200 and spaced apart from the upper end of the variable dilution tank 200, and a column member 341 having an upper end coupled to the lower surface of the support plate 342 and a lower end coupled to the upper surface of the upper jig 110.

The first driving unit 330 may be fixed to the upper surface of the support plate 342 and a hole may be formed in the support plate 342 so that the shaft 320 can penetrate the support plate 342. The shaft 320 may be screwed to the support plate 342. In this case, a screw thread may not be provided in the penetration hole 210.

As shown in FIG. 3, the volume control unit 300 may further include a measuring unit 350 configured to measure a moving distance of the moving unit 310 in the vertical direction.

The measuring unit 350 may include a moving member 351 configured to move in the vertical direction along with the shaft 320, a fixing member 352 coupled to the upper surface of the upper jig 110, and a display member 353 configured to indicate a displacement between the moving member 351 and the fixing member 352.

The moving member 351 may be coupled to the shaft 320 so as to be rotatable, and may move in the vertical direction together with the shaft 320. For example, a stopper (not shown) may be provided on the shaft 320 so that the moving member 351 can move along with the shaft 320 in the vertical direction.

Specifically, the moving member 351 may include a first moving member 351a coupled to the shaft 320 so as to be rotatable, a second moving member 351b protruding to the side of the first moving member 351a, and a third moving member 351c having an upper end coupled to the second moving member 351b and a lower end inserted into the fixing member 352.

For example, the third moving member 351c and the fixing member 352 may be provided in a cylindrical shape, and the third moving member 351c may be inserted into the fixing member 352. A scale may be displayed on the display member 353 so that the moving distance of the moving member 351 can be measured.

As described above, on the upper surface of the upper jig 110, a second hole for inserting a punching needle into the gas diffusion space 130 may be formed, and as shown in FIG. 5, the punching unit 400 may include a punching needle housing 410 coupled to the upper surface of the upper jig 110 with a lower end covering the second hole and configured to accommodate the punching needle inside, a rod 420 with a lower end coupled to the punching needle inside the punching needle housing 410, a second driving unit 430 coupled to an upper end of the rod 420 and configured to move the rod 420 in the vertical direction, and a fixing unit 440 configured to fix a position between the punching needle housing 410 and the second driving unit 430.

The fixing unit 440 may include a first fixing plate 441 having an upper surface to which the lower end of the second driving unit 430 is coupled, and a second fixing plate 442 having a lower surface to which the punching needle housing 410 is coupled, a fixing column 443 fixing the first fixing plate 441 and the second fixing plate 442 such that the distance between the first fixing plate 441 and the second fixing plate 442 is maintained constant.

On the first fixing plate 441 and the second fixing plate 442, a hole through which the rod 420 is penetrated may be formed.

Although embodiments according to the present disclosure have been described above, they are only illustrative and those skilled in the art will understand that various modifications and embodiments of equivalent range are possible therefrom. Therefore, the true technical protection scope of the present disclosure should be defined by the following claims.

Explanation of Symbols 100 . . . Gas diffusion chamber, 110 . . . Upper jig, 111 . . . First hole, 120 . . . Lower jig, 121 . . . Battery accommodating groove, 122 . . . Gas delivery flow path, 130 . . . Gas diffusion space, 200 . . . Variable dilution tank, 210 . . . Penetration hole, 220 . . . Sealing cover, 230 . . . Dilution space, 300 . . . Volume control unit, 310 . . . Moving unit, 320 . . . Shaft, 330 . . . First driving unit, 340 . . . Support unit, 341 . . . Column member, 342 . . . Support plate, 350 . . . Measuring unit, 351 . . . Moving member, 351a . . . First moving member, 351b . . . Second moving member, 351c . . . Third moving member, 352 . . . Fixing member, 353 . . . Display member, 400 . . . Punching unit, 410 . . . Punching needle housing, 420 . . . Rod, 430 . . . Second driving unit, 440 . . . Fixing unit, 441 . . . First fixing plate, 442 . . . Second fixing plate, 443 . . . Fixing column

A gas collecting apparatus of the present disclosure may be capable of collecting a gas with a constant concentration in a gas collecting container by providing a controllable variable space to a gas diffusion space.

The gas collecting apparatus of the present disclosure may provide an improved gas concentration to an analysis device by controlling a space in which gas generated from a battery is diffused in the process of collecting the gas.

1. A gas collecting apparatus comprising:
a gas diffusion chamber including a gas diffusion space;
a variable dilution tank connected to the gas diffusion chamber and including a dilution space;
a volume control unit configured to control a volume of the dilution space of the variable dilution tank;
a punching unit configured to form a perforated hole in a case of a battery insertable into the gas diffusion space of the gas diffusion chamber; and
a gas delivery flow path connected to the gas diffusion chamber to receive gas diffused into the gas diffusion space and deliver the gas to a gas collecting container or a gas analyzer.

2. The gas collecting apparatus of claim 1, wherein the volume control unit comprises:
a moving unit located inside the variable dilution tank and configured to move in a vertical direction;
a shaft coupled to an upper surface of the moving unit, wherein a lower end of the shaft is inserted inside the variable dilution tank through a penetration hole on an upper surface of the variable dilution tank, the shaft configured to move in the vertical direction; and
a first driving unit configured to rotate the shaft with the vertical direction as a rotation axis.

3. The gas collecting apparatus of claim 2, wherein the gas diffusion chamber comprises:
a lower jig with a battery accommodating groove on an upper surface of the lower jig as the gas diffusion space; and
an upper jig coupled to the upper surface of the lower jig while covering the battery accommodating groove, and
wherein a first hole is on an upper surface of the upper jig,
a lower end of the variable dilution tank is coupled to the upper surface of the upper jig while covering the first hole, and
gas in the gas diffusion space and gas in the dilution space are mutually ventilated through the first hole.

4. The gas collecting apparatus of claim 3, wherein the volume control unit further comprises a support unit configured to fix a relative distance between the first driving unit and the upper jig.

5. The gas collecting apparatus of claim 3, wherein the volume control unit further comprises a measuring unit configured to measure a moving distance of the moving unit in the vertical direction.

6. The gas collecting apparatus of claim 5, wherein the measuring unit comprises:
a moving member configured to move in the vertical direction along with the shaft;
a fixing member coupled to the upper surface of the upper jig; and
a display member configured to indicate a displacement between the moving member and the fixing member.

7. The gas collecting apparatus of claim 2, wherein an inner space of the variable dilution tank has a column shape extending in the vertical direction,
a side surface of the moving unit is in contact with an inner circumferential surface of the variable dilution tank, and
the dilution space is a space located at a lower side of the moving unit in the inner space of the variable dilution tank.

8. The gas collecting apparatus of claim 7, wherein a sealing cover surrounding the shaft is located at an upper side of the moving unit in the inner space of the variable dilution tank, and
the sealing cover is contracted or expanded in the vertical direction.

9. The gas collecting apparatus of claim 8, wherein an upper end of the sealing cover is coupled to a ceiling surface of the variable dilution tank in the inner space of the variable dilution tank, and
a lower end of the sealing cover is coupled to the upper surface of the moving unit.

10. The gas collecting apparatus of claim 3, wherein a second hole for inserting a punching needle into the gas diffusion space is on the upper surface of the upper jig, and
the punching unit comprises:
a punching needle housing coupled to the upper surface of the upper jig with a lower end of the punching needle housing covering the second hole and configured to accommodate the punching needle inside;
a rod with a lower end of the rod coupled to the punching needle inside the punching needle housing;
a second driving unit coupled to an upper end of the rod and configured to move the rod in the vertical direction; and
a fixing unit configured to fix a position between the punching needle housing and the second driving unit.
